(19) United States
(12) Patent Application Publication
(10) Pub. No.: US 2026/0292322 A1
(43) Pub. Date: Sep. 24, 2026

(54) MEDICAL INSTRUMENTATION UTILIZING NARROWBAND IMAGING

(71) Applicant: NSV, Inc., Allentown, PA (US)

(72) Inventors: Soham Pathak, Allentown, PA (US); Sharvari Harsh Dalal, Munster, IN (US); Katherine Anne Yanushefski, Zionsville, PA (US)

(21) Appl. No.: 19/682,856

(22) Filed: May 20, 2026

Related U.S. Application Data
(63) Continuation-in-part of application No. 17/441,839, filed on Apr. 3, 2020.
(60) Provisional application No. 62/829,078, filed on Apr. 4, 2019.

(51) Int. Cl. H04N 23/56; A61B 3/12; A61B 3/14; G02B 23/24; H04N 23/50

(52) U.S. Cl. CPC H04N 23/56; A61B 3/12; A61B 3/14; G02B 23/2461; H04N 23/555

(57) ABSTRACT

An illumination device for medical scopic instrumentation is disclosed. The illumination device uses first and second illumination sources positioned at emission apertures around a centrally disposed photoreceiving element aperture, the illumination sources oriented so that light emitted thereby forms converging light cones that overlap at a target examination area within an anatomical region of interest (ROI) to provide uniform, shadow-reduced illumination. The device remains external to the patient body while imaging internal ROIs such as the cervix, and the illumination sources can comprise narrowband LEDs at wavelengths selected for hemoglobin absorption peaks (e.g., ~540 nm green and ~415 nm blue) to enhance vasculature contrast, with optional white light and computational imaging/AI processing of captured images to improve diagnostic accuracy.

### CROSS-REFERENCE TO RELATED APPLICATIONS

This application is a Continuation-in-Part of U.S. patent application Ser. No. 17/441,839, filed on Sep. 22, 2021, which is a national stage entry under 35 U.S.C. § 371 of International Application No. PCT/US 2020/026589, filed on Apr. 3, 2020, which claims priority to U.S. Provisional Patent Application No. 62/829,078, filed on Apr. 4, 2019. The entire disclosures of above-identified applications are hereby incorporated herein by reference.

### TECHNICAL FIELD

The present invention relates to improving medical instrumentation that utilizes visual imaging of a region of interest and, more particularly, to the utilization of narrowband light sources emitting at specific, predefined wavelengths to enable the viewing (and capture) of high contrast digital images without the use of filtered white light.

### BACKGROUND OF THE INVENTION

There are several types of medical procedures that utilize image analysis of selected specimens to aid in the development of a proper diagnosis by evaluating parameters of interest (POI). POI for dermatoscopy, for example, may utilize an analysis of lesion texture, topology, or specific pigmentation characteristics associated with melanocytes in determining a diagnosis. POI for colposcopy, for example, is known to extensively utilize analysis of vascular patterns, lesion size, punctation and identification of squamocolumnar junction (SCJ) in evaluating a patient's condition. POI for fundus imaging may utilize analysis of at least one of retinoblastoma, diabetic retinopathy, and ocular melanoma. These are three specific areas of the use of imaging analysis in the field of medicine. More generally, the foregoing exemplary tests, parameters, and imaging analyses are illustrative only, and the present disclosure encompasses any other medical testing methods, diagnostic procedures, imaging modalities, and corresponding parameters, whether known now or developed in the future, that may be used to evaluate a specimen or patient condition.

Dermatoscopes include a magnifying optical system, a light source illuminating the region to be examined (with hopefully as few reflections as possible), and a power supply for providing electrical energy to the light source. During a medical examination, the dermatoscope is normally placed with a contact plate made of glass on the skin, which is then observed through the optical system. In certain embodiments, dermatoscopic oil or another liquid having a glass-like refractive index is placed between the skin and the dermatoscope, or the contact plate. Some embodiments make use of polarized illumination, since some medical diagnoses are only possible if the region to be examined is viewed under specialized lighting configurations.

An optical colposcope comprises a binocular microscope with a built-in white light source and objective lens attached to a support mechanism. Various levels of magnification are often necessary to detect and identify certain POI indicative of the presence of more advanced pre-cancerous or cancerous lesions. During a colposcopic exam, acetic acid and iodine solutions are usually applied to the surface of the cervix to improve the visualization of abnormal areas. In colposcopy, abnormality of cervical tissue is often assessed with what is known as the “Swede score”. This score specifically takes into account crucial characteristics of cervical tissue such as vessel patterns, which can be assessed and deemed to fall into one of three categories: (1) “fine and regular”; (2) “absent”; or (3) “course or atypical”. In some cases, different-colored filters are used to accentuate blood vessel patterns that cannot be easily seen by using regular white light. This type of vasculature imaging is also useful when viewing oral mucosa and submucosa for the presence of premalignant lesions associated with various oral cancers.

Fundus imaging refers to the acquisition of high-resolution images of the interior surface of the eye, including the retina, optic disc, macula, and posterior pole, for diagnostic and monitoring purposes. Conventional fundus cameras employ optical systems with illumination and focusing mechanisms to capture two-dimensional retinal images. Recent advancements integrate digital sensors, adaptive optics, and image processing algorithms to enhance resolution, reduce artifacts, and enable wide-field visualization.

However, since there is no standard wavelength or spectral bandwidth defined for these filters, different clinical settings may apply “green filters” that transmit so-called green light at different wavelengths, perhaps with different bandwidths. The use of such filters can produce less effective images in some cases, or lead to less consensus between different images of differing qualities. Additionally, green filters placed over white light inevitably diminish the transmission of light, and captured images often appear darker than they should.

In recent times, advances in digital imaging and various software/algorithmic techniques related to imaging have improved the quality of the images in these endeavors and reduced the need to use polarized light or certain filters to capture images. While considered an advance in the state-of-the-art, these techniques are applied subsequent to the process of creating and storing the images. A need remains to improve the quality, resolution, and detail of the images created in the first instance.

### SUMMARY OF THE INVENTION

The need remaining in the prior art is addressed by the present invention, which relates to digital imaging for vasculature analysis and, more particularly, to the utilization of light sources emitting at specific, predefined wavelengths to enable narrowband digital imaging without the use of filters.

In accordance with the present invention, it is proposed to eliminate the use of color-based filters and, instead, provide an illumination source comprised of individual light emitting diodes (LEDs) specifically formed to operate at the wavelengths of interest (e.g., “green”, “blue”, “red”, “yellow”, etc.) based on the absorption spectrum of certain biomolecule(s) of interest present in the region of the body being examined. Advantageously, LEDs may be configured to generate a high intensity, narrowband beam that is well-suited for these medical imaging purposes where the ability to provide a proper diagnosis relies on the ability to create a high contrast image for review by the medical professionals.

In one exemplary embodiment, the present invention takes the form of an illumination source useful in performing digital imaging in conjunction with medical scopic instrumentation. The illumination source comprises at least one narrowband LED operating at a first center wavelength λ₁ associated with a first absorbance peak of a biomolecule present in an anatomical region of interest (ROI) under study, and perhaps another narrowband LED operating at a second center wavelength λ₂ associated with a second absorbance peak of either the same or a different biomolecule(s) present in the anatomical region of interest (ROI) under study (if the biomolecule in the ROI has two separate absorbance peaks, for example, hemoglobin). The LEDs are controlled in a manner that enhances the contrast between a specific set of features in the ROI and surrounding material, enabling the generation of a high-contrast digital image of the ROI.

The inventive illumination source may also include a conventional white light source that is used as before for general observation purposes, with the one or more narrowband LEDs activated when there is a need to create a high contrast image of a particular ROI. The turning “on” and “off” of the narrowband LEDs may be controlled by the individual performing the examination, with LED(s) at the first wavelength energized at a specific time when there is a need to capture a high contrast image (and other LED(s)) perhaps energized at another point in time during the examination. The captured high contrast images may be digitized and stored for analysis at a later point in time, by an individual at a remote location, or the like.

In an aspect, an illumination device for performing digital imaging in conjunction with medical scopic instrumentation is disclosed. The illumination device includes a photoreceiving element positioned at a centrally disposed aperture of the illumination device to receive reflected light from an anatomical region of interest (ROI) under study and create a digital image therefrom. The illumination device further includes a first illumination source positioned at a first emission aperture of the illumination source. The first illumination source is configured to emit light along a first emission axis extending at a first angle relative to an optical axis of the medical scopic instrumentation. The illumination device further includes a second illumination source positioned at a second emission aperture of the illumination source and configured to emit light along a second emission axis extending at a second angle relative to the optical axis. The first emission axis and the second emission axis are oriented so that light emitted by the at least one first illumination source and light emitted by the at least one second illumination source form converging light cones that overlap at a target examination area within the ROI, thereby defining an overlapping illumination zone. The overlapping illumination zone provides substantially uniform light distribution across the target examination area so as to reduce shadow artifacts and enhance visualization of anatomical features in the ROI.

In an embodiment, the illumination device remains external to the body of the subject while the photoreceiving element also remains external to the body of the subject while capturing images of the ROI.

In an embodiment, the illumination device and the photoreceiving element are integrated as part of a single device. In an alternative embodiment, the illumination device and the photoreceiving element are provided as separate, physically distinct devices that are operatively coupled to one another.

In an embodiment, the first angle and the second angle are within a range from 0 degrees to 45 degrees relative to the optical axis.

In an embodiment, a distance from at least one of the at least one first illumination source and the at least one second illumination source to a focal plane associated with the target examination area is within a range from 200 millimeters to 500 millimeters, with a distance of approximately 300 millimeters.

In an embodiment, the at least one first illumination source and the at least one second illumination source are disposed on opposing sides of the centrally-disposed aperture of the illumination device so as to be substantially symmetric about the optical axis.

In an embodiment, at least one of the first angle and the second angle is adjustable to permit a non-symmetric arrangement of the first emission axis and the second emission axis relative to the optical axis.

In an embodiment, the ROI comprises a cervix having a diameter of approximately 25 millimeters, and wherein the illumination device is configured to accommodate a cervical diameter in a range from 20 millimeters to 35 millimeters for a non-pregnant patient and up to about 50 millimeters for a pregnant patient.

In an embodiment, the ROI comprises an anatomical region other than the cervix, including but not limited to an oral cavity, oropharynx, skin surface, or any other anatomical site accessible via a natural orifice or external surface, and the illumination device is configured to accommodate the dimensions of the applicable ROI.

In an embodiment, the illumination device further includes at least one additional illumination source positioned at an emission aperture around the centrally-disposed aperture, a light field produced by the at least one additional illumination source at least partially overlapping the overlapping illumination zone produced by the at least one first illumination source and the at least one second illumination source.

In an embodiment, the illumination device further includes a control unit configured to energize the at least one first illumination source and the at least one second illumination source in a sequence of illumination modes including illumination with only the at least one first illumination source, illumination with only the at least one second illumination source, and simultaneous illumination with both the at least one first illumination source and the at least one second illumination source.

In an embodiment, the control unit is further configured to independently adjust intensities of the at least one first illumination source and the at least one second illumination source to maintain substantially uniform illumination across the ROI while compensating for differences in tissue reflectance.

In an embodiment, the control unit is configured to synchronize the activation of the at least one first illumination source and the at least one second illumination source with an exposure window of the photoreceiving element to prevent spectral banding during sequential wavelength illumination.

In an embodiment, the at least one first illumination source comprises at least one narrowband first wavelength illumination source positioned at the first emission aperture and specifically formed to emit narrowband light at a first center wavelength λ₁ associated with a first absorbance peak of the ROI. The at least one second illumination source comprises at least one narrowband second wavelength illumination source positioned at the second emission aperture and specifically formed to emit narrowband light at a second center wavelength λ₂ associated with a second absorbance peak of the ROI. In an embodiment, the first center wavelength λ₁ and the second center wavelength λ₂ are different from one another. In an alternative embodiment, the first center wavelength λ₁ and the second center wavelength λ₂ are substantially the same, such that both illumination sources operate at the same wavelength simultaneously, for example to achieve uniform, shadow-reduced illumination of the ROI without spectral differentiation. Reflected light at each of the first center wavelength λ₁ and the second center wavelengths λ₂ is directed through the centrally-disposed aperture and into the photoreceiving element for creating a first digital image and a second digital image, respectively, each exhibiting a high contrast between normal and abnormal areas in the ROI.

In an embodiment, the first center wavelength λ₁ is approximately 540 nanometers corresponding to green light and the second center wavelength λ₂ is approximately 415 nanometers corresponding to blue light.

In an embodiment, the illumination device further includes a white light source positioned at an emission aperture selected from the group consisting of the first emission aperture, the second emission aperture, and a third emission aperture for alternative illumination of the ROI. The white light source is operated, independently to the operation of the at least one first illumination source and the at least one second illumination source, to emit white illumination for general inspection.

In an embodiment, the illumination device is utilized in conjunction with a scopic system for viewing vasculature and the first and second center wavelengths are selected to be proximate to absorbance peaks of hemoglobin.

In an embodiment, the illumination device is utilized in conjunction with a dermatoscope and the first center wavelength and the second center wavelength are selected to be proximate to absorbance peaks of skin pigments.

In an embodiment, the first center wavelength is selected from the group consisting of approximately 540 nm, approximately 555 nm, and approximately 575 nm. The second center wavelength is selected from the group consisting of approximately 415 nm, approximately 430 nm, and approximately 450 nm.

In an embodiment, the at least one first illumination source and the at least one second illumination source are narrowband Light Emitting Diodes (LEDs), and wherein each narrowband LED exhibits a full width half maximum (FWHM) of no greater than 30 nanometers. In an alternative embodiment, the at least one first illumination source and the at least one second illumination source comprise narrowband laser light sources configured to emit light at the respective first and second center wavelengths, each laser light source exhibiting a spectral linewidth suitable for the diagnostic imaging application, with eye safety requirements addressed in accordance with applicable standards for the intended use.

In an embodiment, each of the at least one first illumination source and the at least one second illumination source comprises a plurality of LEDs, the plurality of LEDs being disposed at a corresponding plurality of emission apertures around the centrally disposed aperture so that light from the plurality of LEDs collectively contributes to the overlapping illumination zone.

In an embodiment, the at least one first illumination source and the at least one second illumination source comprise narrowband laser light sources configured to emit light towards the ROI.

In an embodiment, the at least one first illumination source is a narrowband LED and the at least one second illumination source is a narrowband laser light source.

In an aspect, a method of acquiring and processing medical images of an anatomical region of interest (ROI) using medical scopic instrumentation is disclosed. The method includes positioning the medical scopic instrumentation so that an optical axis of the medical scopic instrumentation is directed toward the ROI. The method further includes illuminating the ROI with an illumination device comprising at least one first illumination source positioned at a first emission aperture and at least one second illumination source positioned at a second emission aperture. The at least one first illumination source and the at least one second illumination source being oriented so that light emitted thereby forms converging light cones that overlap at a target examination area within the ROI to define an overlapping illumination zone. The method further includes capturing at least one digital image of the ROI with a photoreceiving element positioned at a centrally disposed aperture of the illumination source under illumination provided by the overlapping illumination zone. The method further includes processing the at least one captured digital image using a computational image enhancement engine and an artificial intelligence-based analysis engine.

In an embodiment, the artificial intelligence-based analysis engine comprises a machine learning model trained to classify parameters of interest (POI) in the ROI. The POI comprises at least one of POI for dermatoscopy, POI for colposcopy, POI for fundus imaging, and any other medical testing methods, diagnostic procedures, imaging modalities, and corresponding parameters. Processing the at least one captured digital image comprises automatically generating a clinical metric based on the classified POI.

In an embodiment, the POI for dermatoscopy may utilize an analysis of lesion texture, topology, or specific pigmentation characteristics associated with melanocytes in determining a diagnosis. The POI for colposcopy may utilize analysis of vascular patterns, lesion size, punctation and identification of squamocolumnar junction (SCJ) in evaluating a patient's condition. POI for fundus imaging may utilize analysis of at least one of retinoblastoma, diabetic retinopathy, and ocular melanoma.

Other and further embodiments and features of the present invention will become apparent during the course of the following discussion and by reference to the accompanying drawings.

### BRIEF DESCRIPTION OF THE DRAWINGS

Referring now to the drawings, where like elements include like reference numbers in several views:

FIG. 1 depicts examples of medical instrumentation used to perform optical imaging;

FIG. 2 is a simplified isometric view of an exemplary illumination source formed in accordance with the present invention;

FIG. 3 is a block diagram side view of the illumination source of FIG. 2;

FIG. 4 is a front view of an exemplary arrangement of narrowband LEDs within the inventive illumination source;

FIG. 5 is a front view of an alternative arrangement of narrowband LEDs within the inventive illumination source;

FIG. 6 shown yet another arrangement of narrowband LEDs within an illumination source formed in accordance with the principles of the present invention;

FIG. 7 is a photographic reproduction of a prior art digital image captured with white light;

FIG. 8 is a photographic reproduction of the same ROI as shown in FIG. 7, in this case illuminated with narrowband LEDs of a particular wavelength associated with an absorbance peak of the biomolecule(s) present in the ROI;

FIG. 9 illustrates a front view of the illumination source;

FIG. 10 illustrates a side view of the illumination source;

FIG. 11 illustrates a method for acquiring a digital image of the ROI using the external illumination device and scopic system;

FIG. 12 illustrates a front view of an alternative configuration of the illumination source; and

FIG. 13 illustrates a configuration in which the illumination device and the photoreceiving element are provided as separate.

### DETAILED DESCRIPTION

As mentioned above, clear, high-contrast images of selected specimens are vital for diagnostic impressions, particularly when performing pre-cancer and cancer screening. In accordance with the principles of the present invention, it is proposed to use narrowband light sources, operating at specific pre-determined wavelengths, to produce extremely high contrast images of the portion of the anatomy under study (that is, the “region of interest” or ROI).

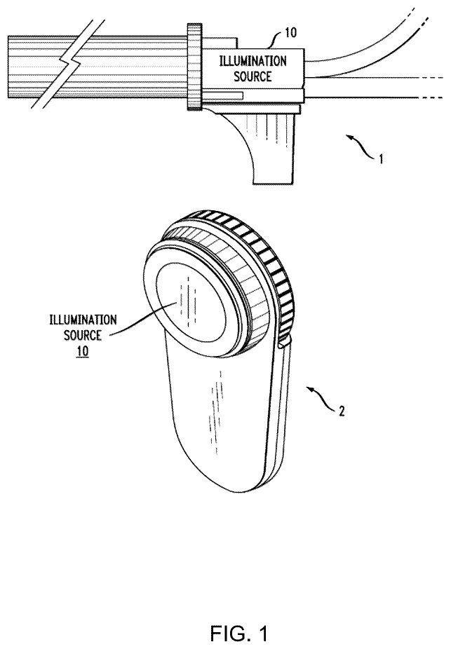

FIG. 1 illustrates exemplary types of medical instrumentation that are used to perform optical imaging and include an illumination source that may be formed to include the LED-based system of the present invention. In particular, FIG. 1 depicts a side view of an exemplary colposcope 1, used in the examination of the cervix (e.g., to study the vasculature system of the cervix). While the specific instrument shown in FIG. 1 is rather compact (and thus portable), many colposcopy systems are large combinations of elements situated in an examination room. An exemplary dermatoscope 2 is also shown in FIG. 1. This type of medical instrumentation is used to view the skin (often with some type of oil or lotion applied to the surface of the skin before bringing the dermatoscope in contact.

Medical instrumentation such as that shown in FIG. 1 is typically based upon the use of a “white light” (full visible spectrum) source to aid the medical professional performing the examination to clearly see the “region of interest” (referred to hereinafter as the “ROI”). It has been known for years, however, that light at certain wavelengths can assist in improving the visualization of blood vessels, skin pigments, mucous, and the like. For example, imaging the cervix with “green” or “blue” light has been found to produce higher contrast images of the underlying vasculature than illumination with white light, since the absorbance spectrum of hemoglobin (a major component of the vessels) includes peaks in the visible part of the spectrum at wavelengths of about 415 nm (“blue” filtered light) and about 540 nm (“green” filtered light). Similar green/blue filters are also used in the study of oral mucosa and submucosa for the presence of premalignant lesions. Abnormal lesions or melanocytes on the surface of the skin (or in the tissue layers immediately beneath the surface) may be better distinguished by using “red” filtered light (a wavelength of about 625 nm) or “yellow” filtered light (a wavelength of about 580 nm).

In the prior art, the medical imaging apparatus utilized various “color” filters in combination with the standard white light source to alter the color of the ROI. As mentioned above, since there is no standard wavelength or spectral bandwidth defined for these filters, different clinical settings may apply “green” filters (using “green” as just one example) that transmit so-called green light at different wavelengths, perhaps with different bandwidths. Moreover, many of these filters may be wideband devices (e.g., bandwidths over 50 nm) that are too broad in spectral response to create an image that clearly delineates boundaries between normal and abnormal tissue. As a result, the use of such filters may produce less effective images in some cases, or lead to less consensus between different images of differing qualities. Additionally, the utilization of these filters in combination with a white light source inevitably diminishes the intensity of the transmitted beam, and captured images often appear darker than they should.

In accordance with the principles of the present invention, it is proposed to eliminate the use of such color-based filters and, instead, provide an illumination source comprised of individual light emitting diodes (LEDs) specifically formed to operate at the wavelengths of interest (e.g., “green”, “blue”, “red”, “yellow”, etc.). Advantageously, LEDs may be configured to generate a high intensity, narrowband beam that is well-suited for these medical imaging purposes where the ability to provide a proper diagnosis relies on the ability to create a high contrast image for review by the medical professionals.

When used as an illumination source for a colposcope, the inventive LED-based source utilizes one or more LEDs that emit at specifically-defined wavelengths that are referenced as “green” and “blue”. The green and blue wavelengths emitted by the LEDs is absorbed by the vessels, while being reflected by the surrounding tissue that lacks hemoglobin. This increases the contrast with which vessels appear in the image. The narrower the bandwidth of the blue and green light (i.e., bandwidths on the order of about 30 nm, or perhaps less) around hemoglobin's absorbance peaks, the greater is the contrast of the vessels in the resulting image. The high contrast between the tissues and vessels significantly improves the visualization of blood vessel patterns, where certain patterns are a known indicator of tissue abnormality. Therefore, the ability to create (and thereafter store) digital images with this level of clarity is a vital need for diagnostic impressions of pre-cancer and cancer (for studying oral mucosa and submucosa as well).

As will also be discussed below, inasmuch as the two different wavelengths penetrate to a different depth within the ROI, by controlling the sequence of illumination for these LEDs (e.g., a “green” exposure, followed by a “blue” exposure), variations in the vasculature at different levels within the tissue may be discerned, providing a “three-dimensional” imaging result.

When used as an illumination source for a dermatoscope, the wavelengths for “red” and “yellow” light beams are known to coincide with the absorbance peaks of medically-relevant pigments (e.g., melanocytes).

In accordance with the principles of the present invention, the number of separate LEDs used, as well as their relative placement within the illumination source, provides the ability to individually manipulate the brightness of the narrowband illumination such that high quality, high contrast images are captured with sufficient brightness and clarity.

In a specific embodiment of the present invention, a scopic diagnostic tool is utilized to illuminate a particular ROI with a collection of illumination sources operating at specific, well-defined wavelengths. In many cases, a first set of LEDs (all operating at a first defined wavelength λ₁) and a second set of LEDs (all operating at a second defined wavelength λ₂) are used as part of the imaging system for these scopes. The LEDs are particularly selected to exhibit a narrow bandwidth to produce a high contrast result, particularly to aid in delineating the boundary between normal and abnormal areas within the ROI. For example, LEDs operating at a “green” wavelength of λ₁∞540 nm that exhibit a full-width-half-maximum (FWHM) of 30 nm, and LEDs operating at a “blue” wavelength of λ₂∞415 nm that exhibit a FWHM of 12 nm can be used, where the FWHM is a well-understood figure of merit defining the distance from a given center wavelength where the output emission drops below half of the maximum emission value. The center wavelength of a given LED is preferably maintained within a narrow range to ensure that images collected using different instruments will be of similar quality.

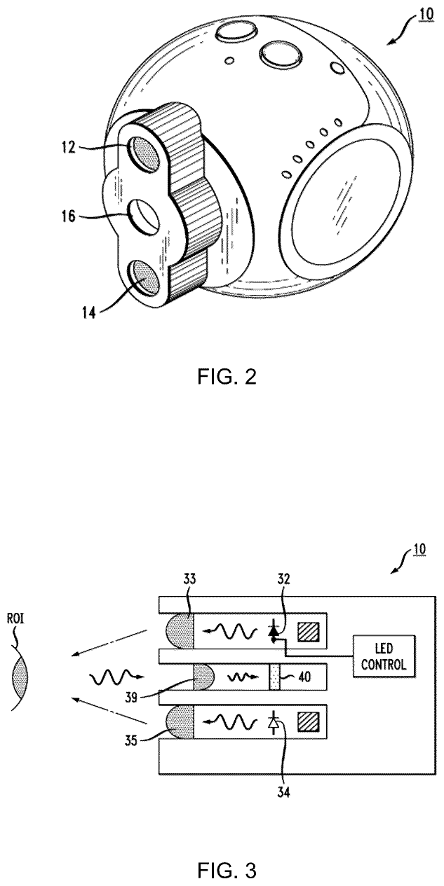

FIG. 2 is a simplified isometric view of an exemplary illumination source 10 formed in accordance with the present invention to be utilized within medical instrumentation such as that illustrated in FIG. 1. In this particular configuration, illumination source 10 is formed to include a pair of opposing apertures 12, 14 through which a narrowband beam from the included LEDs is emitted and directed to an ROI. A central aperture 16 includes a photodetecting arrangement that captures the return light from the ROI. For example, the photodetecting arrangement may take the form of a CCD camera or, preferably, a CMOS detector with appropriate filtering to block stray light outside of the LED wavelengths. As will be discussed in detail below, one or more LEDs may be located at each aperture 12 and 14 (with a white light source in most cases co-located with the LEDs). Additional apertures may be disposed at different locations around the periphery of central aperture 16 to allow for multiple sets of LEDs to be used for narrowband imaging in accordance with the principles of the present invention.

FIG. 3 is a block diagram side view of an exemplary configuration of illumination source 10, in this illustration shown as being used in association with a particular ROI. In this example, a first narrowband LED 32 (operating at a first specifically-defined wavelength λ₁) is positioned in alignment with aperture 12. When illumination source 10 is part of a colposcopic system, first narrowband LED 32 may be a “green” LED, emitting at a center wavelength λ₁∞540 nm, with a FWHM value of 30 nm. When illumination source 10 is part of a dermatoscope, first narrowband LED 32 may be a “red” LED, emitting at a center wavelength λ₁∞625 nm, with a FWHM value of 16 nm. Lensing elements 33 are positioned beyond the output from first LED 32 and used to enable the focusing of the narrowband output from first LED 32 toward the ROI.

Also shown in FIG. 3 is a second narrowband LED 34, operating at a second specifically-defined wavelength and positioned behind aperture 14 of instrumentation 10. When illumination source 10 is part of a colposcope, second narrowband LED 34 may be a “blue” LED, emitting at a center wavelength λ₂∞415 nm, with a FWHM value of 12 nm. When illumination source 10 is part of a dermatoscope, second narrowband LED 34 may be a “yellow” LED, emitting at a center wavelength λ₂∞580 nm, with a FWHM value of 22 nm. Lensing elements 35 are positioned beyond the output from second LED 34 and used to enable the focusing of the narrowband output from second LED 34 toward the ROI.

A conventional white light source 31 is also shown in FIG. 3, where it is to be understood that the inclusion of white light source 31 is optional, but preferable, since in most cases the medical instrumentation would utilize white light source 31 to illuminate the ROI for a portion of an examination and then energize narrowband LEDs 32, 34 as necessary. Indeed, the turning “on” and “off” of LEDs 32 and 34 is typically under the control of the individual performing the examination, allowing for the capture of high contrast images at specific points in time during the examination procedures. As mentioned above, the activation of the narrowband LEDs may be controlled such that the first-wavelength LEDs 32 are energized for a period of time, and then the second-wavelength LEDs 34 are energized for a different period of time, where the separate activation may provide additional imaging clarity of subsurface elements associated with the different depths penetrated by the different wavelengths.

A photoreceiving element 40 is shown in FIG. 3 as positioned behind central aperture 16, with lensing elements 39 disposed at the entrance of photoreceiving element 40. In accordance with the optical imaging properties of medical instruments, the illumination reflected back towards illumination source 10 from the ROI is captured by photoreceiving element 40 and processed using various types of analysis, well known (and also currently evolving) in the art. Photoreceiving element 40 may comprise, for example, a CCD-based camera or a CMOS detector with appropriate wavelength filtering.

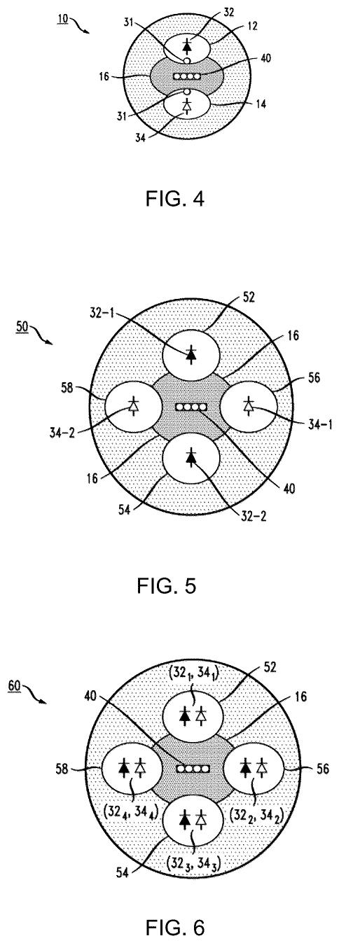

FIG. 4 is front view of the particular arrangement of LEDs 32 and 34 as shown in FIG. 3. FIG. 5 is a front view of an alternative illumination system 50 utilizing pairs of apertures disposed around central aperture 16. In this particular arrangement a first aperture 52 is positioned at the 0° location around the circular form of illumination system 50, with a second aperture 54 located at the 180° position. A second pair of apertures is disposed orthogonal to apertures 52 and 54, with one aperture 56 located at the 90° position and a remaining aperture 58 located at the 270° position. In this particular configuration, first-wavelength (λ₁) LEDs 32-1 and 32-2 are disposed behind apertures 52 and 54 (respectively), and second-wavelength (λ₂) LEDs 34-1 and 34-2 are disposed behind apertures 56 and 58 (respectively).

FIG. 6 shows yet a different arrangement. Here, an illumination system 60 maintains the same set of four apertures 52, 54, 56 and 58 as shown in FIG. 5, but in this case is configured to use (λ₁,λ₂) pairs of LEDs at each of the four quadrant locations as defined above with respect to the arrangement of FIG. 5. A first pair is identified as (LED 32₁, LED 34₁); a second pair is identified as (LED 32₂, LED 34₂); a third pair is identified as (LED 32₃, LED 34₃); and a fourth pair is identified as (LED 32₄, LED 34₄).

In each of these embodiments, a specific switching sequence may be used to control the illumination of the separate LEDs, where as mentioned above it is typically the individual performing the examination who controls when the LEDs are turned “on” and “off”. However, it is to be understood that a computer-based control of LED sequencing may also be implemented in certain applications.

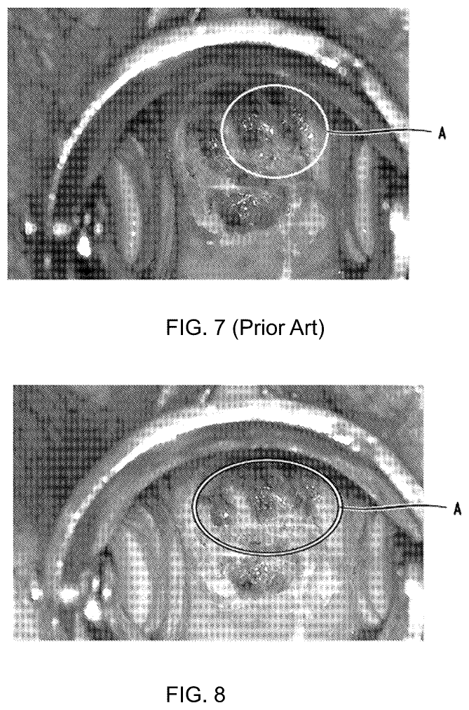

The ability of the narrowband, wavelength-specific LEDs to provide a higher quality, sharper image of an exemplary ROI is shown by comparing a photographic reproduction of a prior art digital image displayed in FIG. 7 (captured using a traditional white light source) to the digital image displayed in FIG. 8, which was captured using green LEDs as an illumination source in accordance with the teachings of the present invention. The higher contrast result of FIG. 8 is evident in the detailed vasculature of the ROI, particularly in comparative regions A (for example).

In addition to the embodiments described above, the disclosure further provides an illumination source as described herein is incorporated into a medical scopic system that employs a dual-illumination source or multi-illumination source architecture configured to generate overlapping light cones at a region of interest (ROI). The overlapping light cones define an overlapping illumination zone at a target examination area and are arranged to provide substantially uniform, shadow-reduced illumination while maintaining high light intensity at the ROI. In the multi-illumination source architecture, multi-LED source may be provided as the first illumination source 12 and the second illumination source 14. Based on the requirement, same wavelength may be turned on for each illumination source. In an alternate implementation, the first illumination source 12 may be set to emit at a wavelength different from the second illumination source 14.

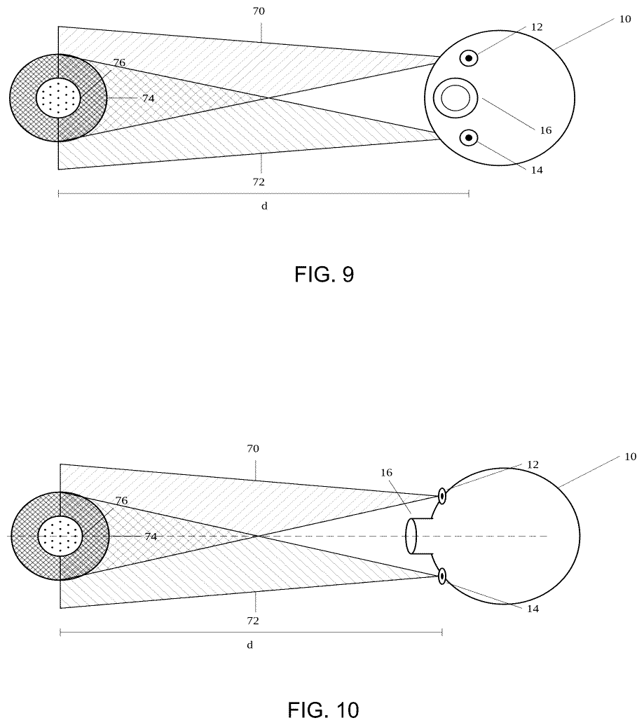

For better understanding, one or more embodiments of these additional embodiments shall be described with respect to FIG. 9 and FIG. 10.

FIG. 9 illustrates a front view and FIG. 10 illustrates a side view of an illumination source 10. The illumination source 10 (also referred to as an illumination device 10) may be mounted at a distal end of a scope that remains external to a patient's body while imaging a region of interest (ROI) 74, such as the cervix. The illumination source 10 includes a first illumination source 12 and a second illumination source 14 disposed on a front face of the illumination device 10 at locations spaced from a central aperture 16 through which an optical axis extends. The central aperture 16 carries imaging optics and a photoreceiving element, as described above, which receives reflected light from the ROI 74 and forms digital images therefrom.

In an embodiment, the first illumination source 12 and the second illumination source 14 are narrowband Light Emitting Diodes (LEDs), and wherein each narrowband LED exhibits a full width half maximum (FWHM) of no greater than 30 nanometers. In an alternative embodiment, the first illumination source 12 and the second illumination source 14 comprise narrowband laser light sources configured to emit light at the respective first and second center wavelengths, each laser light source exhibiting a spectral linewidth suitable for the diagnostic imaging application, with eye safety requirements addressed in accordance with applicable standards for the intended use.

In yet another embodiment, each of the first illumination source 12 and the second illumination source 14 comprises a plurality of LEDs, the plurality of LEDs being disposed at a corresponding plurality of emission apertures around the centrally disposed aperture so that light from the plurality of LEDs collectively contributes to the overlapping illumination zone.

As shown in FIGS. 9 and 10, the first illumination source 12 and the second illumination source 14 are oriented so as to emit light along respective emission axes that are tilted inward toward the optical axis. In the side view of FIG. 10, these emission axes define respective illumination cones 70 and 72 that originate at the illumination sources 12 and 14 and extend toward the ROI 74. The angular orientation of illumination sources 12 and 14 with respect to the optical axis is selected so that cones 70 and 72 converge and overlap in front of the device, forming an overlapping illumination region 76 that substantially spans the diameter of the ROI 74.

The distance d between the illumination source 10 (more particularly the illumination sources 12 and 14) and the ROI 74 represents a typical working distance of the device during examination. In certain embodiments, the distance d is nominally about 300 mm, with an allowable range from approximately 200 mm to approximately 500 mm, so that the overlap of cones 70 and 72 occurs at or near the focal plane of the device corresponding to the surface of the ROI 74. For cervical imaging, the ROI 74 may have a typical diameter of about 25 mm, with a size range from about 20 mm to about 35 mm for non-pregnant patients and up to about 50 mm for pregnant patients, and the positions and angles of illumination sources 12 and 14 are chosen to provide overlapping illumination across at least this range.

In a preferred configuration, illumination sources 12 and 14 are disposed substantially symmetrically about the centrally-disposed aperture 16 on the face of illumination source 10, so that illumination cones 70 and 72 are mirror-symmetric with respect to the optical axis of the device. This symmetry produces a balanced overlapping illumination region and reduces directional shadows that might otherwise obscure portions of the ROI 74. In other embodiments, one or both of the emission axes of illumination sources 12 and 14 may be adjustable with respect to the optical axis, allowing a clinician or system designer to intentionally implement a slightly non-symmetric configuration while still maintaining a substantial overlap between cones 70 and 72 at the ROI 74.

Although FIGS. 9 and 10 depict a pair of illumination sources 12, 14, the illumination source 10 can optionally include additional illumination sources positioned around the centrally-disposed aperture 16, each generating a respective illumination cone that at least partially overlaps the region defined by cones 70 and 72. In such multi-illumination source embodiments, the combined effect of the overlapping cones yields a highly uniform illumination field over ROI 74 while enabling flexible control of intensity, wavelength, and spatial distribution of light.

In a further embodiment, each of the first and second illumination sources 12 and 14 comprises a multi-LED array configured to be selectable among a plurality of wavelengths to provide uniform, shadow-reduced illumination of the ROI. In such an embodiment, the first illumination source 12 may be set to emit at any one of a predefined set of wavelengths, and the second illumination source 14 may independently be set to emit at any one of a predefined set of wavelengths, including the same wavelength or a different wavelength from the first illumination source. The control unit is configured to select the operating wavelength for each illumination source independently.

In one embodiment, the illumination source 10 is mounted to a colposcope or other optical scope that remains external to the body of a patient while imaging an internal anatomical structure such as the cervix. The colposcope defines a central optical axis extending from the objective toward the cervical surface, and the illumination source 10 is arranged around the optical axis so that illumination is directed toward a cervical ROI through a natural orifice.

The illumination source 10 includes a photoreceiving element positioned at the centrally-disposed aperture 16 to receive reflected light from the ROI 74 and to create one or more digital images therefrom, as described for the narrow-band embodiments above. The first illumination source 12 is positioned at a first emission aperture of the illumination source 10 and is configured to emit light along a first emission axis that extends at a first angle relative to the optical axis. The second illumination source 14 is positioned at a second emission aperture of the illumination source and is configured to emit light along a second emission axis that extends at a second angle relative to the optical axis.

As described above, the first and second emission axes are oriented such that light emitted by the first illumination source 12 and light emitted by the second illumination source 14 form converging light cones that overlap at the cervical surface or other target examination area. The overlap of the light cones defines an overlapping illumination zone that is configured to span at least the field of view (FOV) of the scopic instrumentation at the applicable working distance, ensuring that providing substantially uniform light distribution across the ROI and minimizing shadows that would otherwise be cast by cervical topography or instruments in the field. In the context of cervical imaging, this overlapping illumination zone enhances visualization of parameters of interest (POI). POI for dermatoscopy, for example, may utilize an analysis of at least one of lesion texture, topology, or specific pigmentation characteristics associated with melanocytes in determining a diagnosis. POI for colposcopy, for example, is known to extensively utilize analysis of at least one of vascular patterns, lesion size, punctation, and identification of squamocolumnar junction (SCJ) in evaluating a patient's condition, such as assessment of pre-cancerous and cancerous changes. POI for fundus imaging may utilize analysis of at least one of retinoblastoma, diabetic retinopathy, and ocular melanoma.

In some embodiments, the first and second angles are selected within a range from approximately 0 degrees to approximately 45 degrees with respect to the optical axis. For example, illumination sources 12 and 14 may be tilted inward from the optical axis so that the respective light cones intersect at or near the focal plane of the colposcope, which is positioned at the cervical surface. A typical working distance from the illumination sources 12 and 14 to the focal plane may be approximately 300 mm, with a preferred range from about 200 mm to about 500 mm to accommodate different scope geometries and clinical setups.

The physical separation between the first and second emission apertures can be selected based on scope head diameter, desired cone angle, and the size of the ROI 74. In one cervical application, the ROI 74 has a typical diameter of about 25 mm, and can vary from about 20 mm to about 35 mm for non-pregnant patients and up to about 50 mm for pregnant patients. The positions and angles of the illumination sources are therefore chosen to provide overlap across at least this range of cervical diameters so that the entire cervix is illuminated uniformly for most patients.

In certain embodiments, the first illumination source 12 and the second illumination source 14 are arranged substantially symmetrically about the optical axis, for example at diametrically opposite positions around the centrally-disposed aperture 16 of the photoreceiving element. Symmetric placement provides an “ideal” configuration for eliminating directional shadows and producing a visually balanced image of the ROI 74. However, the illumination source 10 may also be configured so that one or both of the first and second angles are adjustable, thereby allowing a controlled non-symmetric configuration to be used for specific imaging tasks or anatomical conditions without limiting the scope of the invention to strictly symmetric arrangements.

Although many examples describe two primary illumination sources 12 and 14, the illumination source 10 can employ two or more illumination sources, positioned at multiple emission apertures around the centrally-disposed aperture 16. Multiple illumination sources can be grouped into sets, with each set producing a corresponding light field that at least partially overlaps with light fields from other sets, further improving spatial uniformity of illumination and providing flexible patterns for illumination control.

The dual-illumination source and multi-illumination source configurations described above can be implemented using the narrow-band spectral characteristics described in connection with the parent embodiments. In one embodiment, the first illumination source 12 comprises a narrowband first-wavelength illumination source centered at approximately 540 nm (green), and the second illumination source 14 comprises a narrowband second-wavelength LED centered at approximately 415 nm (blue), with each LED selected to exhibit a full-width half maximum (FWHM) of no greater than about 30 nm. For example, a blue LED may have an FWHM of about 12 nm, while a green LED may have an FWHM of about 30 nm, so that each wavelength band aligns with an absorbance peak of hemoglobin and enhances vessel-to-tissue contrast in the resulting images.

In addition to blue and green narrow-band LEDs, at least one LED can be configured to emit white light for conventional visualization, with the white light source positioned at one of the multiple emission apertures, (e.g. the first emission aperture, the second emission aperture, or at a third emission aperture). The system can thus switch between white illumination for general inspection and narrow-band illumination for enhanced contrast imaging. For other applications, such as dermatoscopy or oral cavity imaging, LEDs at alternative wavelengths (for example, red, yellow, or other visible or near-infrared bands) can be selected based on absorption and scattering characteristics of pigments or tissues relevant to the specific diagnostic task, while still employing the overlapping-cone geometry described above.

In various embodiments, the center wavelengths used for the first and second LEDs 12, 14 are not limited to a single green and a single blue wavelength, but may be selected from predefined sets tailored to particular tissues and diagnostic tasks. By way of example, a first center wavelength associated with the first LED 12 can be selected from approximately 540 nm, approximately 555 nm, and approximately 575 nm, each of which corresponds to strong absorption features in hemoglobin or related chromophores in submucosal vasculature. Likewise, a second center wavelength associated with the second LED 14 can be selected from approximately 415 nm, approximately 430 nm, and approximately 450 nm, which are suitable for exciting fluorescence and enhancing contrast in superficial epithelial structures and lesions. In one oral-imaging embodiment, the first center wavelength is approximately 545 nm, selected to emphasize hemoglobin absorption in submucosal vasculature, while the second center wavelength is approximately 405 nm, selected to excite porphyrin fluorescence in oral lesions and thereby increase the apparent contrast between diseased and surrounding tissue. These wavelength ranges are exemplary and can be adjusted or combined with other bands as needed for specific clinical applications, while still employing the overlapping-cone illumination geometry described herein.

Although some embodiments depict a single first LED 12 and a single second LED 14, in practice the illumination source 10 can be implemented as a group of discrete narrowband LEDs. For example, each of the first and second LEDs may comprise a plurality of LEDs, with each LED of the group positioned at a corresponding emission aperture around the centrally disposed aperture so that light from the plurality of LEDs collectively defines the effective first or second illumination source. The individual LED emission cones from a given group at least partially overlap one another and also overlap the emission cones of the other group, so that the aggregate effect of all LEDs is to generate the overlapping illumination zone at the target examination area with improved spatial uniformity and controllable intensity.

A control unit associated with the illumination source 10 is configured to energize LEDs individually or in groups to achieve different illumination modes, including: illumination with only the first LED 12 or LED set, illumination with only the second LED 14 or LED set, simultaneous illumination with both sets, and sequences involving white light and one or more narrow-band wavelengths. The control unit can independently modulate LED intensity to maintain substantially uniform brightness across the ROI 74, compensating for variations in tissue reflectivity, patient anatomy, or LED aging, while preserving the desired overlapping-cone effect at the focal plane.

In an embodiment, the control unit coordinates operation of the first LED 12 and the second LED 14 with an exposure window of the photoreceiving element such that illumination at different wavelengths is delivered only during a time interval in which the photoreceiving element is actively acquiring image data. By synchronizing the activation of the LEDs with the exposure window, the control unit reduces or prevents spectral banding and related image artifacts that may otherwise arise when sequential wavelength illumination is unsynchronized with the sensing operation of the photoreceiving element. The control unit may be further configured to switch between the first LED 12 and the second LED 14 in a temporally controlled sequence, for example on a per-frame or per-line basis, so that each wavelength component is emitted during a corresponding exposure period. In this manner, the photoreceiving element receives wavelength-specific illumination under substantially consistent sensing conditions, thereby improving spectral uniformity, image fidelity, and the reliability of captured spectral information during sequential wavelength illumination.

In contrast to endoscopes or other devices that place illumination and imaging components inside a bodily lumen, the embodiments described herein maintain the colposcope or other scope external to the patient's body. The illumination source and photoreceiving element are mounted on or integrated with an external scope head, and the optical axis extends from the scope through a natural orifice to the internal ROI, such as the cervix. This configuration allows the system to provide narrow-band, overlapping-cone illumination and digital imaging capabilities without inserting the scope into the body cavity, thereby preserving patient comfort and allowing easier integration into standard colposcopy workflows.

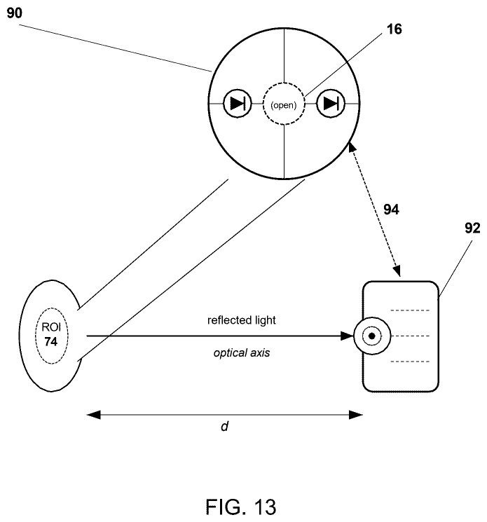

In an alternative embodiment, the illumination device 10 and the photoreceiving element 40 are provided as separate, physically distinct devices (see FIG. 13). In such a configuration, the illumination device 10 is positioned to direct illumination toward the ROI, and the photoreceiving element 40 is independently positioned to receive reflected light from the ROI and create digital images therefrom. The illumination device 10 and the photoreceiving element 40 are operatively coupled, for example via wired or wireless communication, so that illumination timing and image capture can be coordinated. Both the illumination device 10 and the photoreceiving element 40 remain external to the body of the subject during examination. This configuration may be advantageous in clinical settings where flexibility in device positioning is required or where the illumination device 10 and imaging system are provided by different manufacturers or integrated into different equipment platforms.

Similar external configurations can be used for imaging other anatomical regions accessible via natural orifices or external surfaces, such as the oral cavity, pharynx, or skin. For each application, the illumination source angles, working distance, and wavelength selections can be adjusted while maintaining the general architecture in which multiple illumination sources produce converging light cones that overlap at the ROI and the scope remains external to the body.

It is to be noted that as mentioned above an exemplary LED-based illumination source formed in accordance with the present invention most likely also includes the standard white light illumination source, as it is still important to capture various other details of the ROI. In an exemplary procedure, for example, a white light illumination source may be used for most of examination, with the narrowband LED-based illumination source activated (as controlled by the clinician, perhaps) during specific periods of time when the vasculature, skin pigmentation, mucosa, or the like, need to be imaged in detail.

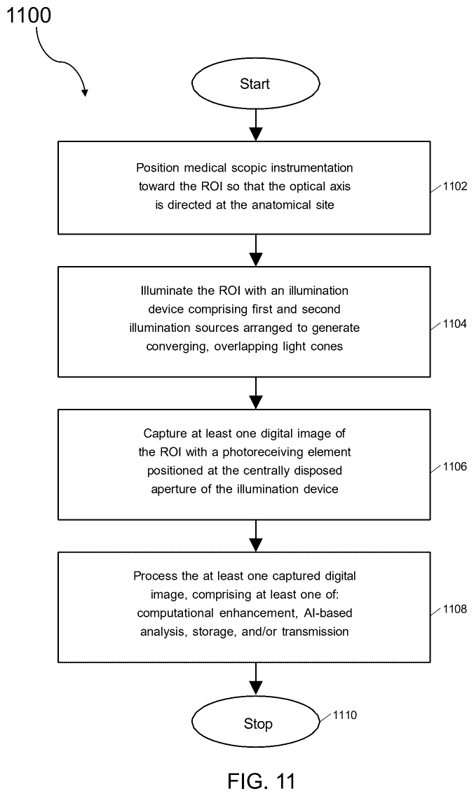

FIG. 11 illustrates a method 1100 for acquiring a digital image of the ROI using the external illumination device and scopic system described in the invention.

In operation 1102, the method starts by positioning medical scopic instrumentation toward the ROI so that the optical axis of the scope is directed at the anatomical site to be examined, such as a cervix, oral cavity, skin region, or any other suitable anatomical site. In operation 1104, the ROI is illuminated with the illumination device that includes the first and second illumination sources arranged to generate converging light cones that overlap at the target examination area, thereby creating the overlapping illumination zone that provides substantially uniform, shadow-reduced illumination. In operation 1106, a digital image of the ROI is captured with a photoreceiving element positioned at the centrally disposed aperture of the illumination device while the ROI is under illumination from the overlapping illumination zone. In operation 1108, the at least one captured digital image is processed, wherein processing comprises at least one of computational image enhancement, artificial intelligence-based analysis of the digital image to identify features indicative of normal or abnormal tissue, storage of the digital image for subsequent review, and transmission of the digital image to a remote location for analysis. The method stops at step 1110.

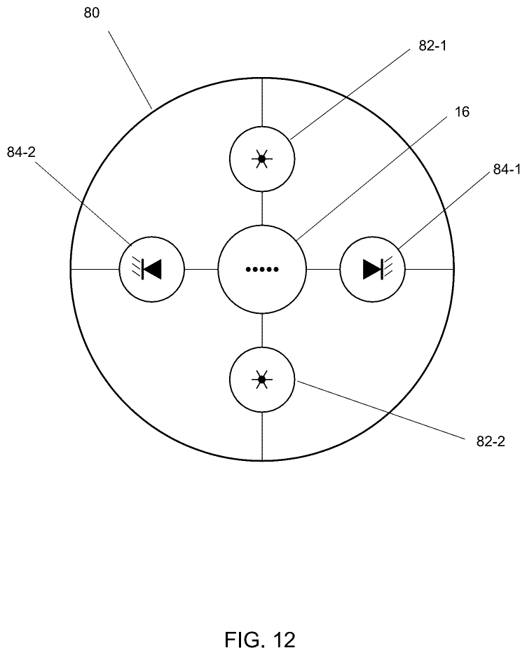

FIG. 12 illustrates a front view of an alternative configuration of an illumination device 80 described in the invention. In this configuration, the illumination device 80 may include illumination sources that are arranged at multiple positions around the centrally-disposed aperture. The illumination sources include white light sources 82 and narrowband illumination sources 84 disposed at respective clock positions around the perimeter of the centrally-disposed aperture.

In an embodiment, the white light sources 82 may include a first white light source 82-1 and a second white light source 82-2. As illustrated in FIG. 12, the first white light source 82-1 is positioned at 12 o'clock and the second white light source 82-2 is positioned at 6 o'clock. Similarly, the narrowband illumination sources 84 may include a first narrowband illumination source 84-1 and a second narrowband illumination source 84-2. As illustrated, the first narrowband illumination source 84-1 is positioned at 3 o'clock and the second narrowband illumination source 84-2 is positioned at 9 o'clock. In an alternate embodiment, the white light sources 82 and the narrowband illumination sources 84 may be positioned at a strategic location on the illumination device 80.

In some embodiments, the plurality of illumination sources comprises a plurality of LEDs, wherein the LEDs may emit the same wavelength or different wavelengths. The LEDs may be selectively activated or deactivated in any suitable combination, such that all of the LEDs are on, all of the LEDs are off, or any subset of the LEDs is on while the remaining LEDs are off. By way of example, where the illumination sources are arranged at respective positions corresponding to the numbers on a clock face, e.g., 1 through 12, any selected group of illumination sources, such as the even-numbered sources, may be activated while the odd-numbered sources are deactivated, or vice versa. More generally, any other combination of on/off states may be employed.

In further embodiments, the plurality of illumination sources may include a combination of LEDs and lasers. The LEDs and lasers may be independently controlled and selectively activated or deactivated in any desired combination, including activation of all sources, activation of only a subset of sources, or any other configuration suitable for the intended illumination pattern.

FIG. 13 illustrates a configuration in which an illumination device 90 and a photoreceiving element 92 are provided separately. As illustrated in FIG. 13, the illumination device 90 and the photoreceiving element 92 are physically distinct devices operatively coupled to one another through an operative coupling 94. In an embodiment, the operative coupling may be a wired connection or a wireless connection.

In such embodiment, the illumination device 90 may emit the narrowband light towards the ROI 74. Further, the reflected light may be received at the photoreceiving element 92 positioned at a distance “d” from the ROI and physically separated from the illumination device 90. The photoreceiving element 92 may transmit the data associated with the reflected light to the illumination device 90 through the operative coupling 94.

In an alternate embodiment, an artificial intelligence-based analysis engine may process the digital image to generate a clinical metric. The digital image may be captured from a fundus imaging apparatus. The artificial intelligence-based analysis engine may comprise a machine learning model trained to classify parameters of interest (POI) in the ROI to generate the clinical metric.

Fundus imaging is a quick, painless, and noninvasive way to photograph the back of the eye, where the retina, optic disc, macula, and retinal blood vessels can be seen. It is widely used in ophthalmology to document the condition of the eye, detect early signs of disease, and monitor changes over time. Because it provides a permanent visual record, clinicians can compare images taken at different visits to assess whether a condition is stable, improving, or worsening. This makes fundus imaging especially useful in diseases such as diabetic retinopathy, glaucoma, age-related macular degeneration, and other retinal disorders.

In general, the descriptions of the details and embodiments of the narrow band illumination system have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the prior art.

What is claimed is:

1. An illumination device for performing digital imaging in conjunction with medical
scopic instrumentation, the illumination device comprising:
a photoreceiving element positioned to receive reflected light from an anatomical region of interest (ROI) under study and create a digital image therefrom;
at least one first illumination source positioned at a first emission aperture of the illumination device and configured to emit light along a first emission axis extending at a first angle relative to an optical axis of the medical scopic instrumentation; and
at least one second illumination source positioned at a second emission aperture of the illumination device and configured to emit light along a second emission axis extending at a second angle relative to the optical axis;
wherein the first emission axis and the second emission axis are oriented so that light emitted by the at least one first illumination source and light emitted by the at least one second illumination source form converging light cones that overlap at a target examination area within the ROI, thereby defining an overlapping illumination zone; and
wherein the overlapping illumination zone provides substantially uniform light distribution across the target examination area.

2. The illumination device as defined in claim 1, wherein the illumination device is positioned externally to a body of a subject during operation.

3. The illumination device as defined in claim 1, wherein the photoreceiving element is positioned at a centrally disposed aperture of the illumination device.

4. The illumination device as defined in claim 1, wherein the photoreceiving element captures images of the ROI while the illumination device remains external to a body of a subject.

5. The illumination device as defined in claim 1, wherein the first angle and the second angle are within a range from 0 degrees to 45 degrees relative to the optical axis.

6. The illumination device as defined in claim 1, wherein a distance from at least one of the at least one first illumination source and the at least one second illumination source to a focal plane associated with the target examination area is within a range from 200 millimeters to 500 millimeters, with a distance of approximately 300 millimeters.

7. The illumination device as defined in claim 1, wherein the at least one first illumination source and the at least one second illumination source are disposed on opposing sides of a centrally-disposed aperture of the illumination device so as to be substantially symmetric about the optical axis.

8. The illumination device as defined in claim 7, wherein at least one of the first angle and the second angle is adjustable to permit a non-symmetric arrangement of the first emission axis and the second emission axis relative to the optical axis.

9. The illumination device as defined in claim 1, wherein the ROI comprises a cervix having a diameter of approximately 25 millimeters, and wherein the illumination device is configured to accommodate a cervical diameter in a range from 20 millimeters to 35 millimeters for a non-pregnant patient and up to about 50 millimeters for a pregnant patient.

10. The illumination device as defined in claim 1, further comprising at least one additional illumination source positioned at an emission aperture around a centrally-disposed aperture, a light field produced by the at least one additional illumination source at least partially overlapping the overlapping illumination zone produced by the at least one first illumination source and the at least one second illumination source.

11. The illumination device as defined in claim 1, further comprising a control unit configured to energize the at least one first illumination source and the at least one second illumination source in a sequence of illumination modes including illumination with only the at least one first illumination source, illumination with only the at least one second illumination source, and simultaneous illumination with both the at least one first illumination source and the at least one second illumination source.

12. The illumination device as defined in claim 11, wherein the control unit is further configured to independently adjust intensities of the at least one first illumination source and the at least one second illumination source to maintain substantially uniform illumination across the ROI while compensating for differences in tissue reflectance.

13. The illumination device as defined in claim 11, wherein the control unit is configured to synchronize activation of the at least one first illumination source and the at least one second illumination source with an exposure window of the photoreceiving element to prevent spectral banding during sequential wavelength illumination.

14. The illumination device as defined in claim 1, wherein:
the at least one first illumination source comprises at least one narrowband first wavelength illumination source positioned at the first emission aperture and specifically formed to emit narrowband light at a first center wavelength λ₁ associated with a first absorbance peak of the ROI;
the at least one second illumination source comprises at least one narrowband second wavelength illumination source positioned at the second emission aperture and specifically formed to emit narrowband light at a second center wavelength λ₂ associated with a second absorbance peak of the ROI; and
reflected light at each of the first center wavelength λ₁ and the second center wavelengths λ₂ is directed through a centrally-disposed aperture and into the photoreceiving element for creating a first digital image and a second digital image, respectively, each exhibiting a high contrast between normal and abnormal areas in the ROI.

15. The illumination device as defined in claim 14, wherein the first center wavelength λ₁ is approximately 540 nanometers corresponding to green light and the second center wavelength λ₂ is approximately 415 nanometers corresponding to blue light.

16. The illumination device as defined in claim 14, further comprising a white light source positioned at an emission aperture selected from the group consisting of the first emission aperture, the second emission aperture, and a third emission aperture for alternative illumination of the ROI.

17. The illumination device as defined in claim 16, wherein the white light source is operated, independently to operation of the at least one first illumination source and the at least one second illumination source, to emit white illumination for general inspection.

18. The illumination device as defined in claim 14, wherein the illumination device is utilized in conjunction with a scopic system for viewing vasculature, and the first center wavelength λ₁ and the second center wavelength λ₂ are selected to be proximate to absorbance peaks of hemoglobin.

19. The illumination device as defined in claim 14, wherein the illumination device is utilized in conjunction with a dermatoscope and the first center wavelength and the second center wavelength are selected to be proximate to absorbance peaks of skin pigments.

20. The illumination device as defined in claim 14, wherein:
the first center wavelength is selected from the group consisting of approximately 540 nm, approximately 555 nm, and approximately 575 nm, and
the second center wavelength is selected from the group consisting of approximately 415 nm, approximately 430 nm, and approximately 450 nm.

21. The illumination device as defined in claim 20, wherein the first center wavelength is substantially same as the second center wavelength.

22. The illumination device as defined in claim 19, wherein:
the first center wavelength is approximately 545 nm corresponding to hemoglobin absorption in submucosal vasculature, and
the second center wavelength is approximately 405 nm corresponding to porphyrin fluorescence excitation in oral lesions.

23. The illumination device as defined in claim 1, wherein the at least one first illumination source and the at least one second illumination source are narrowband Light Emitting Diodes (LEDs), and wherein each narrowband LED exhibits a full width half maximum (FWHM) of no greater than 30 nanometers.

24. The illumination device as defined in claim 1, wherein each of the at least one first illumination source and the at least one second illumination source comprises a plurality of LEDs, the plurality of LEDs being disposed at a corresponding plurality of emission apertures around a centrally-disposed aperture so that light from the plurality of LEDs collectively contributes to the overlapping illumination zone.

25. The illumination device as defined in claim 1, wherein the at least one first illumination source and the at least one second illumination source comprise narrowband laser light sources configured to emit light towards the ROI.

26. The illumination device as defined in claim 1, wherein the at least one first illumination source is a narrowband LED and the at least one second illumination source is a narrowband laser light source.

27. The illumination device as defined in claim 1, wherein the illumination device and the photoreceiving element are integrated as part of a single device.

28. The illumination device as defined in claim 1, wherein the illumination device and the photoreceiving element are provided as separate, physically distinct devices that are operatively coupled to one another.

29. The illumination device as defined in claim 1, wherein the ROI comprises an anatomical region including an oral cavity, oropharynx, skin surface, or any other anatomical site accessible via a natural orifice or external surface.

30. A method of acquiring and processing medical images of an anatomical region of
interest (ROI) using medical scopic instrumentation, the method comprising:
positioning the medical scopic instrumentation so that an optical axis of the medical scopic instrumentation is directed toward the ROI;
illuminating the ROI with an illumination device comprising at least one first illumination source positioned at a first emission aperture and at least one second illumination source positioned at a second emission aperture, the at least one first illumination source and the at least one second illumination source being oriented so that light emitted thereby forms converging light cones that overlap at a target examination area within the ROI to define an overlapping illumination zone;
capturing at least one digital image of the ROI with a photoreceiving element positioned at a centrally disposed aperture of the illumination device under illumination provided by the overlapping illumination zone; and
processing the at least one digital image using at least one of a computational image enhancement engine and an artificial intelligence-based analysis engine.

31. The method as defined in claim 30,
wherein the artificial intelligence-based analysis engine comprises a machine learning model trained to classify parameters of interest (POI) in the ROI; and
wherein processing the at least one digital image comprises automatically generating a clinical metric based on the classified POI.

32. The method as defined in claim 31, wherein the POI comprises at least one of POI for dermatoscopy, POI for colposcopy, POI for fundus imaging and any other medical testing methods, diagnostic procedures, imaging modalities, and corresponding parameters, and wherein:
the POI for dermatoscopy utilizes an analysis of at least one of lesion texture, topology, or specific pigmentation characteristics associated with melanocytes in determining a diagnosis;
the POI for colposcopy utilizes analysis of at least one of vascular patterns, lesion size, punctation and identification of squamocolumnar junction (SCJ) in evaluating a patient's condition; and
the POI for fundus imaging utilizes analysis of at least one of retinoblastoma, diabetic retinopathy, and ocular melanoma.
